(19) United States
(12) Patent Application Publication
(10) Pub. No.: US 2004/0138696 A1
(43) Pub. Date: Jul. 15, 2004

(54) Embolic entrapment sheath

(76) Inventors: William J. Drasler, Minnetonka, MN (US); Mark L. Jenson, Greenfield, MN (US)

Correspondence Address: VIDAS, ARRETT & STEINKRAUS, P.A., 6109 BLUE CIRCLE DRIVE, SUITE 2000, MINNETONKA, MN, 55343-9185, US

(21) Appl. No.: 10/683,866

(22) Filed: Oct. 10, 2003

Related U.S. Application Data
(60) Provisional application No. 60/417,825, filed on Oct. 11, 2002.

(51) Int. Cl. A61M 29/00

(52) U.S. Cl. 606/200

(57) ABSTRACT

An embolic entrapment device and system comprises a flexible polymeric sheath and an expansion member engaged to each end of the sheath. The device is used in a method of treating a lesion by placing the device across the lesion and expanding the device.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

The use of endoprostheses such as stents, stent-grafts, grafts, vena cava filters, embolic filters, etc is well known in maintaining the patency of bodily vessels and treating stenoses within arteries and other body spaces. Typically, a catheter equipped with an angioplasty balloon and/or implantable endoprosthesis is advanced through the vessel to the occlusion site, which is subject to an aneurysm, which has a lesion present or is otherwise damaged. The angioplasty balloon may then be used to reduce the lesion, an endoprosthesis may be positioned across the lesion, or other treatment options may be undertaken.

However, a concern with the use of angioplasty balloons and endoprosthesis implantation is that during or subsequent to such procedures there exists the potential of releasing emboli distally into the vessel with potentially negative effects on the patient, such as ischemic stroke.

To address this concern many devices have been developed to protect against embolization that may occur as a result of advancing, utilizing and/or implanting a medical device into a vessel. For example, the use of fluid permeable filters for trapping and/or removing friable emboli are well known.

It remains desirable however, to provide for an implantable sheath or cover that may be positioned across a lesion of a vessel to cover the lesion and prevent emboli from entering the blood stream as a result of a catheter's passage, the expansion of a balloon, or the delivery of an endoprosthesis to the site.

All U.S. patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

The present invention is directed to a variety of embodiments, in at least one embodiment the invention is directed to an embolic entrapment device that may be implanted across a lesion of a vessel thereby covering the lesion and preventing emboli from entering the blood stream.

In at least one embodiment the device comprises an expandable tubular body or sheath constructed at least partially from one or more flouropolymers, such as for example extruded polytetrafluoroethylene (ePTFE). Some other materials also suitable for use in constructing the sheath are described below. The sheath may constructed of a material that is elastically deformable and/or plastically deformable. In some embodiments the sheath may include one or more shape memory materials, such as a shape memory polymer, to allow or aid in expanding the sheath, or a portion thereof, from an unexpanded state to an expanded state.

In at least one embodiment the sheath has an unexpanded state wherein the material of the sheath is folded into a reduced diameter configuration which may be expanded by a radially outward force supplied by a balloon or other expansion device. In at least one embodiment the sheath is capable of stretching, and/or being stretched, from the unexpanded state to an expanded state.

In at least one embodiment the sheath is characterized as being porous to allow blood and other material to flow freely through the sheath while preventing larger particles of emboli from passing therethrough. The size of the pores or holes may vary but are preferably between about 0 to about 300 microns in width or diameter. In at least one embodiment the pores are about 30 to about 75 microns in diameter.

While various embodiments of the invention may be equipped with a sheath having a wide range of wall thicknesses, it is desirable to provide the sheath with a minimal thickness in order to minimize the profile of the device and optimize performance. Because it is not necessary to provide a sheath that functions to support the vessel in which the device is implanted, it is possible to provide the sheath with an extremely thin wall thickness. In at least one embodiment the sheath has a wall thickness of less than about 0.5 mm. In at least one embodiment the wall thickness of the sheath is about 25 to about 250 microns. In some embodiments, the wall thickness of the sheath will depend on the desired diameter of pores which the sheath material defines.

In at least one embodiment the one or more ends of the sheath include an expander element. An expander element is positioned within or engaged adjacently to the sheath of the device. In some embodiments the expander elements are substantially spring like structures of interconnected struts. The expander elements may be expandable from an unexpanded configuration to an expanded configuration, such as by balloon expansion, and/or the expander elements may be self-expanding.

While it may appear in some embodiments that the expander elements have many features in common with known devices such as stents, it should be noted however, that unlike stents, the expander elements described herein do not provide structural support to the vessel. Instead, the expander elements described herein have structural elements which are suitable to expand the ends of the sheath engaged thereto, but exert a force insufficient to actively support a vessel in the expanded state.

In embodiments where the expander elements, or portions thereof, are self-expandable, at least a portion of the expander element is constructed of an expandable shape memory material such as nitinol and/or shape memory polymer material. The expander elements provide the device with the capability of being self-expandable from a reduced diameter delivery state, where the device may be mounted on a delivery catheter, to an expanded state wherein the device is expanded to enclose a lesion adjacent thereto prior to the implantation of a stent or other device there under.

In embodiments where the expander elements, or portions thereof, are balloon expandable, or externally expandable by some other method or device, the expander elements may include materials such as stainless steel, etc in their construction.

In the various embodiments, the struts of the expander elements have a diameter of about 0.005 to about 0.001 inches in thickness. In some embodiments the expander elements are provided with a minimal number of strut elements.

Where the device has expander elements on both ends, the device will be positioned within the vessel such that the expander elements are respectfully positioned proximally and distally adjacent of the lesion while the sheath is pushed outward against the lesion.

In at least one embodiment of the invention the device, or one or more portions thereof are bioabsorbable. In some embodiments, the device, or portions thereof are constructed such that bioabsorbtion occurs within about a day to about six weeks or more following implantation of the device into a vessel.

Some embodiments of the invention are directed to a system for treating a vessel wherein a catheter is equipped with an embolic entrapment device. In some embodiments the catheter may also be equipped with an angioplasty balloon, a stent, and a mechanism for delivering the stent to a lesion site, to which the embolic entrapment sheath is first deployed.

At least one embodiment of the invention is directed to a method of entrapping a lesion or filtering emboli, wherein an embolic entrapment device is first deployed across the lesion site and subsequently, one or more medical devices such as a stent, stent graft, etc, are delivered into the flow path defined by the device. For example a stent may be deployed within the expanded embolic entrapment device to ensure provide structural support to the vessel and and/or reduce the lesion without fear of emboli entering the blood stream.

These and other embodiments which characterize the invention are pointed out with particularity in the claims annexed hereto and forming a part hereof. However, for a better understanding of the invention, its advantages and objectives obtained by its use, reference should be made to the drawings which form a further part hereof and the accompanying descriptive matter, in which there is illustrated and described embodiments of the invention.

### BRIEF DESCRIPTION OF THE SEVERAL VIEWS OF THE DRAWING(S)

A detailed description of the invention is hereafter described with specific reference being made to the drawings.

FIG. 1 is a perspective side view of an embodiment of the invention.

FIGS. 2-6 show a series of cross-sectional side views of a method, system and apparatus for delivering a device to a lesion site of a body vessel, wherein:

FIG. 2 shows the system being advanced to the lesion site;

FIG. 3 shows the initial delivery of the device to the lesion site;

FIG. 4 shows the expansion of the device about the lesion;

FIG. 5 shows an example of subsequent advancement of a stent through the flow path defined by the device shown in FIG. 4; and

FIG. 6 shows the expanded stent in combination with the device.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

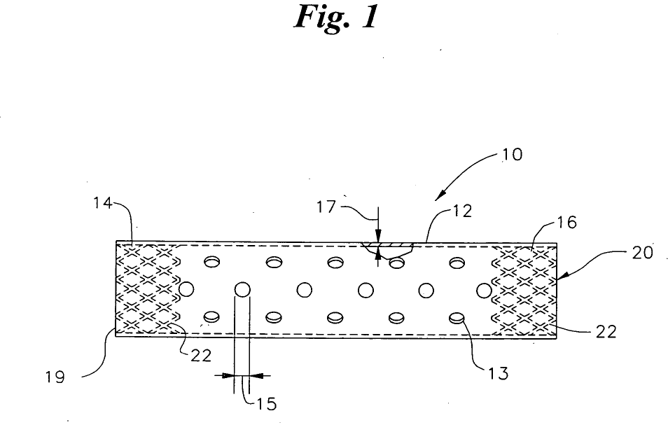

As indicated above the present invention is directed to a variety of embodiments. In FIG. 1 for example, an embodiment of the invention comprises an embolic entrapment device, indicated generally at 10. Device 10 comprises a flexible and expandable sheath 12 of polymeric material such as ePTFE or similar materials. Sheath 12 may include other materials in addition to or instead of ePTFE. Some examples of such materials include but are not limited to other flouropolymers, silicon, polyester, Dacron, polyurethane, polyethylene terephthalate (PET), polyglycol adipate, (PGA), poly(L-lactide) (PLLA), etc.

In some embodiments sheath 12 may be bioabsorbable and/or include surface features or other mechanisms for delivering a drug or other therapeutic agent to a lesion site.

Where the sheath 12 or other portion of the device 10 is bioabsorbable, bioabsorbtion may take place within a day to within about 6 weeks following implantation of the device 10 into the body.

In some embodiments the sheath 12 may be comprised of one or more layers of sheath material or coatings of material. The various layers may have similar or differing physical characteristics such as lubricity, columnar strength, hoop strength, fiber orientation, etc.

As is shown in FIG. 1, in at least one embodiment of the invention, the sheath 12 defines one or more holes or pores 13 having a sufficient size or diameter 15 to allow blood and other desirable material to freely flow through the sheath while preventing passage of the larger particles of undesirable material such as emboli. The size 15 of pores may be between zero to about 300 microns. In some embodiments the size 15 is between about 25 microns to about 75 microns. In at least one embodiment the size 15 of one or more pores 13 is about 50 microns.

The size of the pores 13 may vary depending on the thickness 17 of the sheath wall 19. As indicated above it is desirable to provide the sheath with a minimal thickness in order to minimize the profile of the device and optimize performance. Preferably the thickness 17 is less than about 0.5 mm and is more preferably between about 25 to about 250 microns.

One or both end regions 14 and 16 of sheath 12 are engaged to or disposed about an expander element 20. Expander element 20 is constructed of a plurality of interconnected and/or interwoven struts or members 22 to form a spring like ring or biasing device. In some embodiments the expander elements 20 are constructed of a shape memory metal such as nitinol, and/or others. Other materials suitable for use in constructing expander elements 20 include shape memory polymers. In some embodiments where the expander elements are not self-expandable the elements 20 may be made of any biocompatible or biocompatible coated material such as stainless steel, etc.

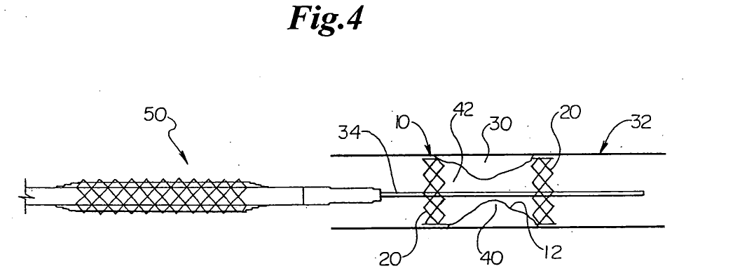

In order to provide an expander that exerts a sufficient amount of force to expand the end of the sheath 12 and/or maintain the ends of the sheath 12 in the expanded state, such as is shown in FIGS. 4-6, but which does not necessarily provide any structural support to the vessel in the expanded state, the struts 22 of the elements 20 are provided with an extremely small diameter. In at least one embodiment the diameter of the struts is about 0.005 to about 0.001 inches. Preferably the elements 20 are provided with only a sufficient number of strut elements necessary to expand the sheath 12 ends and/or maintain the ends in the expanded state.

In some embodiments at least a portion of the device 10, such as expander elements 20, include one or more radiopaque materials to allow the device 10, or portions thereof, to be detectable within the body by fluoroscopy or other techniques.

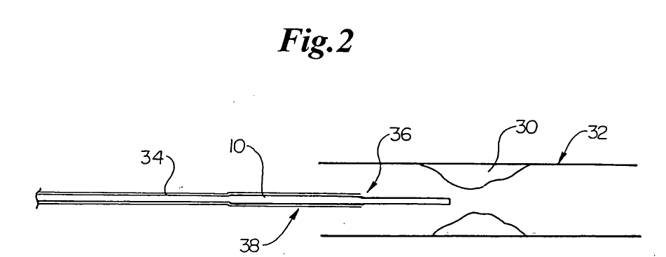

As indicated above, the device 10 is intended for use in entrapping or covering a lesion site 30 within a vessel 32 such as is shown in FIGS. 2-6. In order to position the device 10 across the lesion site 30, the device 10 is advanced to the lesion site 30 on a catheter or guide wire 34, such as in the manner shown in FIG. 2.

In the embodiment shown the device 10 is disposed about a distal portion 36 of a guide wire 34. The device 10 is positioned on the guide wire in a reduced diameter or unexpanded state. In some embodiments where the sheath 12 is plastically deformable, the sheath 12 is provided with a folded configuration to be placed in the unexpanded state. Alternatively, where the sheath 12 is elastically deformable, in the unexpanded state the sheath 12 may be at its nominal diameter which is stretched or otherwise expanded when the device 10 is delivered.

The device 10 is maintained in the unexpanded state by a retractable sheath, sleeve or other device 38.

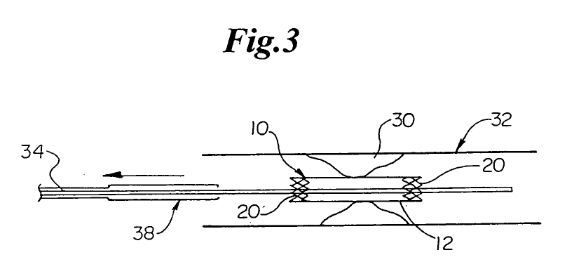

Once the guide wire is advanced to the proper position within the vessel 32 and the device 10 spans the lesion site 30, retractable sleeve 38 is withdrawn and the device 10 is free to expand, such as is shown in FIG. 3.

As is shown in FIG. 4, as the expander elements 20 self-expand, and/or are expanded by a balloon or other device (not shown), the sheath 12 is pushed (or pulled) radially outward, so that the outside surface 40 of the sheath 12 is abutted against the formerly exposed surface of the lesion 30. As a result the lesion is entrapped within the confines of the sheath 12 and the device 10 defines a flow path 42 free of embolic material.

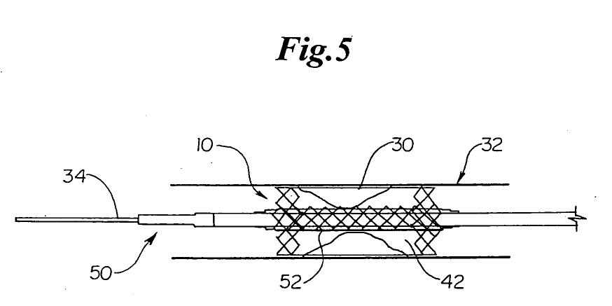

As is shown in FIGS. 4 and 5, once the device 10 is positioned in the manner described above, a medical device 50 such as a balloon equipped catheter, and/or a stent delivery catheter may be advanced along guide wire 34 through the flow path 42 defined by the device 10.

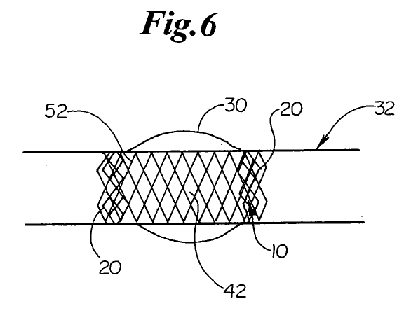

In some embodiments, such as in the examples shown in FIGS. 5 and 6 the device 10 is a part of a system which also include the medical device 50 and/or a stent or other implantable endoprosthesis 52. As part of such a system, once the device 10 is positioned in the manner previously described, the stent 52 is deployed across the lesion site 30 within the flow path 42 defined by device 10.

When the stent or other device 52 is deployed by balloon expansion or other delivery mechanism (such as self-expansion of a stent, where the device 52 is a self-expanding stent), the stent pushed against the device 10, as opposed to the lesion directly. As a result the lesion can be reduced and the flow path 42 enlarged, without the fear of emboli from the lesion entering the vessel.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the preferred and alternate embodiments of the invention. Those skilled in the art may recognize other equivalents to the specific embodiment described herein which equivalents are intended to be encompassed by the claims attached hereto.

1. An embolic entrapment device being expandable from an unexpanded state to an expanded state, the device comprising: a flexible polymeric sheath, and two expansion members, each expansion member being engaged to an end of the sheath.

2. A system for treating a lesion within a body lumen comprising:
a guide wire;
an embolic entrapment device disposed about a portion of the guide wire in an unexpanded state, the device being expandable from the unexpanded state to an expanded state when the device is positioned across a lesion, the device comprising a flexible polymeric sheath, and two expansion members, each expansion member being engaged to an end of the sheath, in the expanded state each expansion member being positioned adjacent to a respective end of the lesion and at least a portion of an external surface of the sheath being in contact with at least a portion of the lesion, in the expanded state the device defining a flow path therethrough;
a stent delivery mechanism movably disposed about the guide wire, when the device is in the expanded state the stent delivery mechanism is constructed and arranged to be advanced along the guide wire and positioned within the flow path of the device, the stent delivery mechanism comprising a stent and a device for delivering the stent, when the stent delivery mechanism is positioned within the flow path of the device, the stent delivery mechanism is constructed and arranged to deliver the stent therein.

3. A method of treating a lesion within a body lumen comprising the steps of:
advancing a guide wire equipped with an embolic entrapment device disposed about a portion thereof through the body lumen to the lesion, the device comprising a flexible polymeric sheath, and two expansion members, each expansion member being engaged to an end of the sheath;
expanding the device from an unexpanded state to an expanded state, such that in the expanded state each expansion member is positioned adjacent to a respective end of the lesion and at least a portion of an external surface of the sheath being in contact with at least a portion of the lesion, in the expanded state the device defining a flow path therethrough;
advancing a stent delivery mechanism along the guide wire to the lesion;
positioning the stent delivery mechanism within the flow path of the device;
delivering a stent from the stent delivery mechanism into the flow path of the device wherein the stent in the delivered state provides structural support to the lumen and applies a compressive force to the lesion.
